(19) United States
(12) Patent Application Publication
(10) Pub. No.: US 2010/0093094 A1
(43) Pub. Date: Apr. 15, 2010

(54) TRIAZINE DENDRIMERS AND METHODS OF MAKING AND USING THE SAME FOR NUCLEIC ACID TRANSPORT

(76) Inventors: Thomas Kissel, Staufen (DE); Olivia M. Merkel, Marburg (DE); Meredith Mintzer, College Station, TX (US); Eric Simanek, College Station, TX (US)

Correspondence Address: BAKER BOTTS L.L.P., 2001 ROSS AVENUE, SUITE 600, DALLAS, TX, 75201-2980, US

(21) Appl. No.: 12/549,182

(22) Filed: Aug. 27, 2009

Related U.S. Application Data
(60) Provisional application No. 61/092,249, filed on Aug. 27, 2008.

(51) Int. Cl. C12N 15/09; C07H 21/04

(52) U.S. Cl. 435/455; 536/23.1; 977/754

(57) ABSTRACT

According to one embodiment, the disclosure relates to a dendrimer complex usable for transfection of a cell and including a nucleic acid to be transfected non-covalently bound to a triazine dendrimer vector. According to another embodiment, the disclosure provides a method of transfecting a cell by contacting the cell with a dendrimer complex as described above. After the contact, the dendrimer complex enters the cell. According to a third embodiment, the disclosure relates to a method of making a triazine dendrimer complex for cell transfection by covalently bonding a series of triazine monomers to a central monomer through a series of reactions to form a triazine dendrimer, then non-covalently bonding a nucleic acid to the dendrimer to form a triazine dendrimer complex operable to transfect a cell.

### PRIORITY CLAIM

The current application claims priority under 35 U.S.C. §119 to U.S. Provision Patent Application No. 61/092,249 filed on Aug. 27, 2008, titled “Synthesis and Physiochemical Evaluation of Melamine-Based Dendrimers for DNA Transfection,” incorporated by reference herein.

### STATEMENT OF GOVERNMENT INTEREST

At least portions of the invention were developed using United States government funding provided through the National Institutes of Health (GM R01 064560) and the National Science Foundation (DGE 0538547). Accordingly, the United States government has certain rights in the invention.

### TECHNICAL FIELD

The current disclosure relates to triazine dendrimers able to bind nucleic acids. These dendrimers may be used to transport bound nucleic acids from the exterior of a cell to the interior. For example, these dendrimers may be used in transfection or gene therapy. The disclosure also relates to methods of making such dendrimers.

### BACKGROUND

The movement of a purified nucleic acid, such as DNA or RNA, or unnatural synthetic analogues, from the external environment into a cell is often referred to as transfection. As a result of transfection, the nucleic acid often exerts an effect in the cell. For example, the DNA may be expressed as a protein altering one or more basic functions in the cell or its interactions with its environment. RNA may block the expression of RNA within the cell already, preventing the expression of a protein. Transfection with certain nucleic acids may kill the cell, which may be very desirable if the cell is, for example, a cancer cell. These various effects may be achieved both in vitro and in vivo, depending on the reason for transfection. For example, in vitro transfection may be sufficient for research purposes, but in vivo or ex vivo transfection may be needed to treat a disease in patient, for example via gene therapy.

Transfection has been traditionally achieved by a variety of mechanisms, but the two primary mechanisms involve the use of either viral or non-viral carriers. In viral transfection, the purified nucleic acid is placed in a virus that is able to inject the nucleic acid into the cell, much as the virus would normally inject its own DNA or RNA. In non-viral transfection, the purified nucleic acid is bound to a vector, typically a chemical, able to move it from the exterior of the cell to the interior of the cell and then allow the nucleic acid to have its desired effect in the cell. The nucleic acid bound to the vector is often referred to as a complex.

The transfection efficiency, which generally describes how well the transported nucleic acid is able to achieve its desired effect, of non-viral vectors is often hindered by three obstacles: the toxicity of the vector, the affinity of the vector/nucleic acid complex for the cell surface, and the stability of the vector/DNA complex. Effective non-viral vectors include cationic liposomes, linear polymers, and certain dendrimers. Polymeric and dendrimeric systems using polyamidoamine (PAMAM) (e.g. SuperFect™, Qiagen, Valencia, Calif.), polypropyleneimine (PPI), and polyethylenimine (PEI) are often effective transfection vectors, although they do not rival viruses in transfection efficiency.

Transfection efficiency may vary with nucleic acid, cell, or organism for particular vectors, creating a need for new vectors to provide alternative transfection vehicles, particularly if these options work better for a given type of transfection. Further, when transfection will occur in an organism, the ability of a vector/nucleic acid complex to be placed in a useful or economical formulation and to appropriately withstand physiological conditions in the organism as well as to be cleared from the organism without inflicting substantial harm may also play a significant role in vector selection. The ease and cost of production as well as the potential for a vector to carry the wrong nucleic acid to a cell may also influence vector choice.

One way in which a vector may be used within an organism, particularly a human patient, is through gene therapy. Gene therapy has gained importance in recent years due to its potential for targeting genetic diseases and inhibiting tumor or cancer growth. However, gene therapy has been limited by the difficulties encountered when trying to deliver foreign DNA to the patient's cells or genome. Both viral and non-viral systems have been developed to overcome various difficulties, but room for improvement remains. For example, viral delivery systems, such as retroviruses, lentiviruses, and adenoviruses, have shown extremely high transfection efficiencies upon reaching a target cell, but are often hindered by the potential for genetic recombination as well as difficulties in scaling-up production to commercial levels. Accordingly, a need for different gene therapy delivery systems exists.

### SUMMARY

The current disclosure provides triazine dendrimers that may be used as vectors for transfection, including transfection that takes place as routine experimentation and as part of gene therapy, among other uses.

According to one embodiment, the disclosure relates to a dendrimer complex usable for transfection of a cell and including a nucleic acid, either natural or unnatural, to be transfected non-covalently bound to a triazine dendrimer vector.

According to another embodiment, the disclosure provides a method of transfecting a cell by contacting the cell with a dendrimer complex as described above. After the contact, the dendrimer complex enters the cell.

According to a third embodiment, the disclosure relates to a method of making a triazine dendrimer complex for cell transfection by covalently bonding a series of triazine subunits to a central core through a series of reactions to form a triazine dendrimer, then non-covalently bonding a nucleic acid to the dendrimer to form a triazine dendrimer complex operable to transfect a cell.

Other alternative embodiments are disclosed below.

### BRIEF DESCRIPTION OF THE DRAWINGS

A more complete and thorough understanding of the present embodiments and advantages thereof may be acquired by referring to the following description taken in conjunction with the accompanying drawings, which represent example embodiments of the disclosure, and wherein:

FIG. 1 illustrates generations one through three of the G dendrimer.

FIG. 2 illustrates the second generation F dendrimer.

FIG. 3 illustrates the second generation B dendrimer.

FIGS. 4A and 4B illustrates peripheral groups that may be attached to triazine dendrimers.

FIG. 5 illustrates guanidinylated peripheral groups that may be attached to triazine dendrimers.

FIG. 6 illustrates comparative data for G, F and B dendrimers from a variety of tests. The G, F, and B dendrimers or dendrimer complexes may also have various peripheral groups attached.

FIG. 7 illustrates a synthesis scheme for the G dendrimer (Scheme 1a), for the F dendrimer (Scheme 1b), and for the B dendrimer (Scheme 1c).

FIGS. 8A-8E illustrate a synthesis scheme for a fragmented form of a fourth generation triazine dendrimer.

FIGS. 9A-9C illustrate a synthesis scheme for an unfragmented form of a fourth generation triazine dendrimer.

FIG. 10 illustrates triazine dendrimer containing a paclitaxel peripheral group.

### DETAILED DESCRIPTION

The current disclosure relates to triazine dendrimers able to bind to nucleic acids including, naturally occurring nucleic acids, purified nucleic acids, and synthetic analogues thereof. The dendrimers may be symmetric or asymmetric, the latter dendrimer also referred to as fragmented triazine dendrimers or star triazine dendrimers. The current disclosure also relates to complexes containing a triazine dendrimer vector and a nucleic acid. Further, the disclosure relates to methods of forming the triazine dendrimers, methods of forming complexes, methods of using complexes to transfect cells, including information that may be used by one of ordinary skill in the art of selecting particular complexes for use in transfecting particular cells.

### Triazine Dendrimers

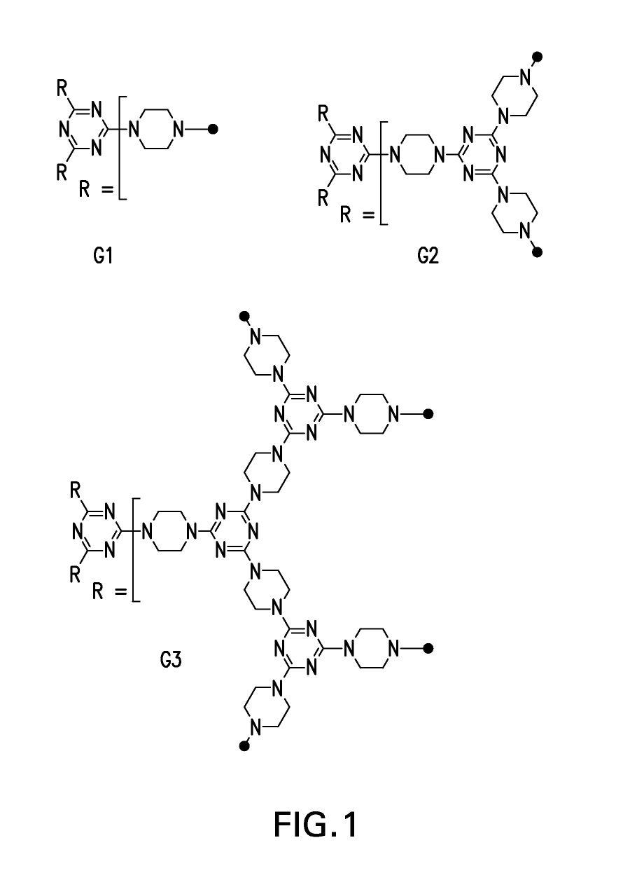

A dendrimer is a branched polymer that contains successive layers of a triazine branching points connected by linker groups that can vary in composition. Dendrimers may be classified by “generation” which identifies the number of layers generally present in the dendrimer. The current specification illustrates one type of dendrimer with the various generations one to three shown in FIG. 1. Throughout this disclosure, the generation of a dendrimer is indicated by a number after the dendrimer's identify letter. For example, a 4th generation version of dendrimer X would be denoted “X4.”

According to some embodiments, other layered triazine multimers may also be included in place of a triazine dendrimer group, such as fractured dendrimers and discrete, asymmetric, branched materials. Throughout the specification, these materials are referred to as dendrimers, although they could suitably described as hyperbranched triazine dendrimers or star triazines dendrimers.

According to one embodiment of the current disclosure, the triazine dendrimer may include dendrimers between one and eight generations. The generation of the dendrimer as well as the addition of various peripheral groups or the amount of nucleic acid added may be adjusted to achieve appropriate bodily clearance, movement into target tissues, or uptake by cells.

Although only specific generations of specific dendrimers are described in detail herein, other dendrimers of all generations may be used in other embodiments of the disclosure.

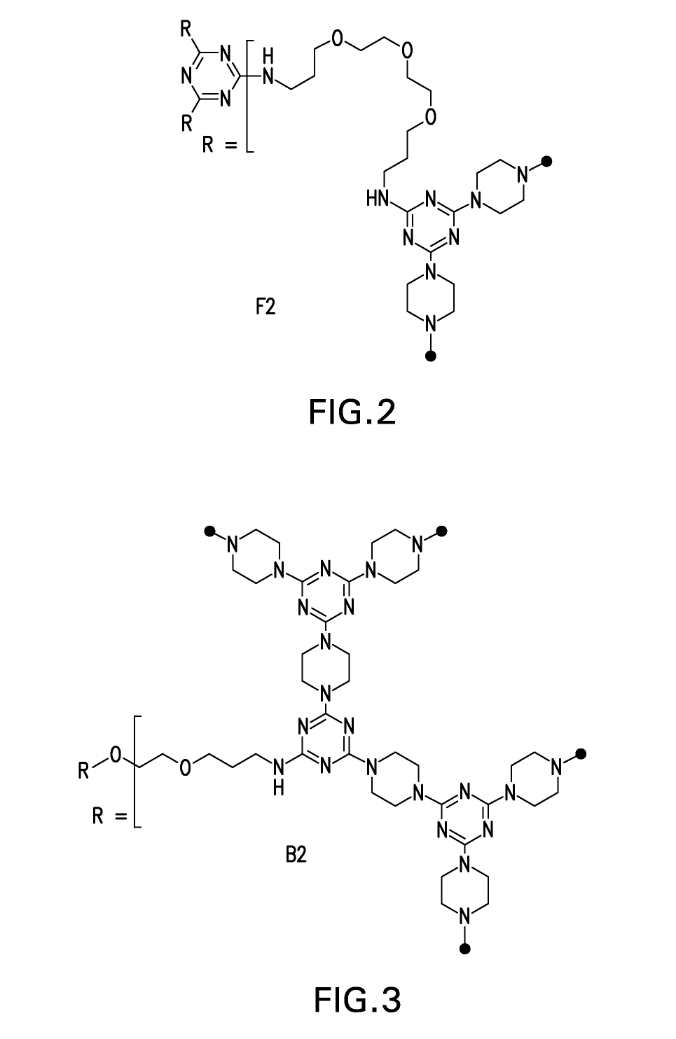

According to another embodiment, the dendrimer may build from a particular triazine. For example, the monomer may include melamine. According to one specific embodiment of the current disclosure, the triazine dendrimer may include rigid, aliphatic linkers such as the diamine piperazine linkers contained in the “G” dendrimers as shown in generations one to three in FIG. 1. According to another specific embodiment, the triazine dendrimer may include flexible, hydrophilic linkers as represented by the dendrimer designated as “F” as show in the second generation in FIG. 2. According to another specific embodiment, the triazine dendrimer may include a bow-tie-shaped dendrimer designated as “B” as shown in the second generation in FIG. 3. This dendrimer uses both types of linkers.

According to other embodiments, peripheral groups may be added to the dendrimer core to achieve various properties or functionalities. For example, it is well known that polycations are often toxic to cells. Accordingly, polycationic dendrimer cores may be modified with peripheral groups that reduce the available cationic groups and improve biocompatibility and decrease toxicity. Polycations are helpful, however, in interacting with the cell membrane to induce endocytosis and for attracting nucleic acids, which are polyanions. Accordingly, the choice of polycation-depleting peripheral groups of the amount of them included in final triazine dendrimer vectors may be adjusted to achieve sufficient DNA binding. In specific embodiments, peripheral groups may include or be derived from neutral groups such as PEG, lipids, aliphatic groups, or hydroxyl groups. The neutrality of these groups may decrease the polycation toxicity while not repelling the polyanionic nucleic acid. Further, the hydrophobic nature of certain dendrimer cores and/or peripheral groups may promote interaction with the cell surface and thus improve transfection.

Although inclusion of only one peripheral group is demonstrated in the specific examples herein to allow more precise investigation of particular groups, in practice one of ordinary skill in the art could readily adapt the synthesis methods disclosed herein to introduce more than one functional group on a single dendrimer. This, for example, may allow one to obtain a dendrimer having certain desirable properties conferred by one functional group while minimizing its undesirable properties, or it may allow one to obtain a dendrimer with multiple desirable properties or exploiting a synergistic effect between multiple peripheral groups in obtaining a single property.

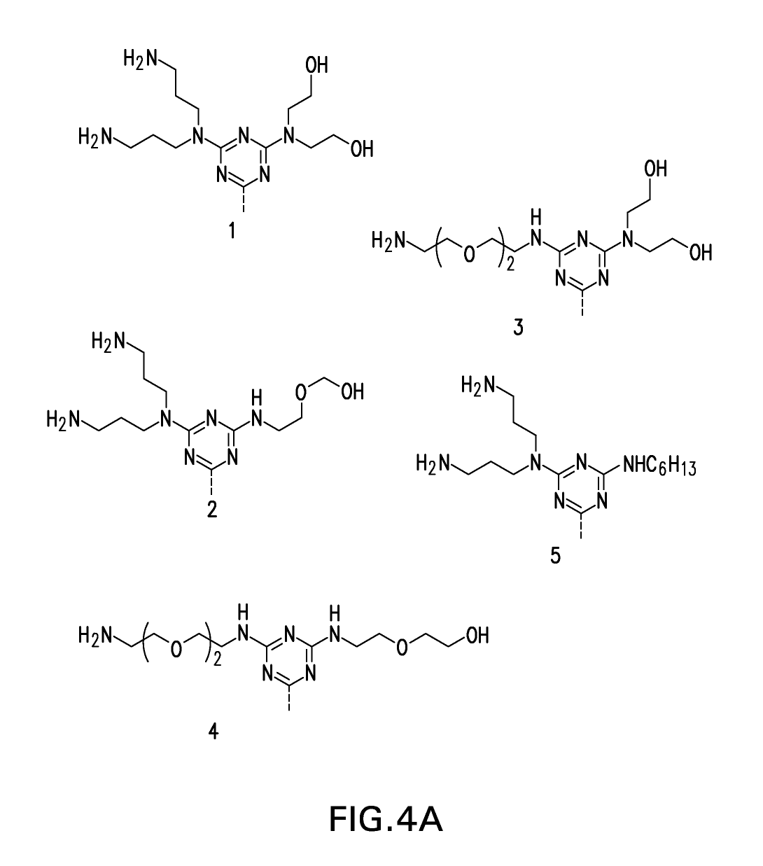

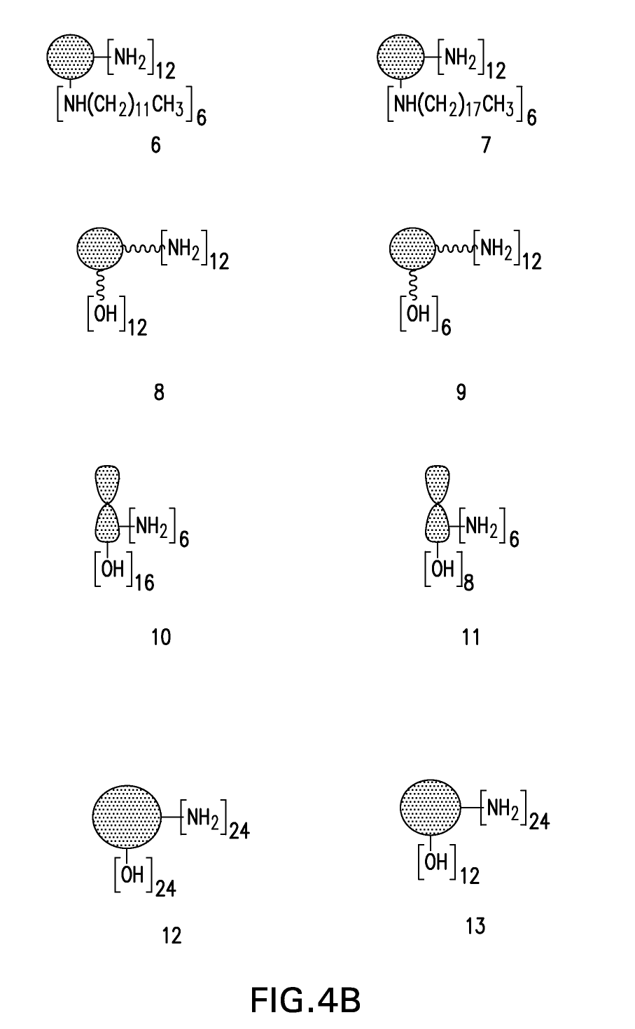

According to specific embodiments, peripheral groups of the current disclosure may include hydroxyl groups, PEG and PEG-like linkers, and alkyl groups. Thirteen specific triazine peripheral groups are shown in FIG. 4. Throughout this specification, the addition of one of these peripheral groups to a triazine dendrimer is designed by a dash followed by the peripheral group number after the dendrimer and generation designation. For example, a fourth generation dendrimer X core with peripheral group 1 would be designated “X4-1.” Other triazine peripheral groups may also be used.

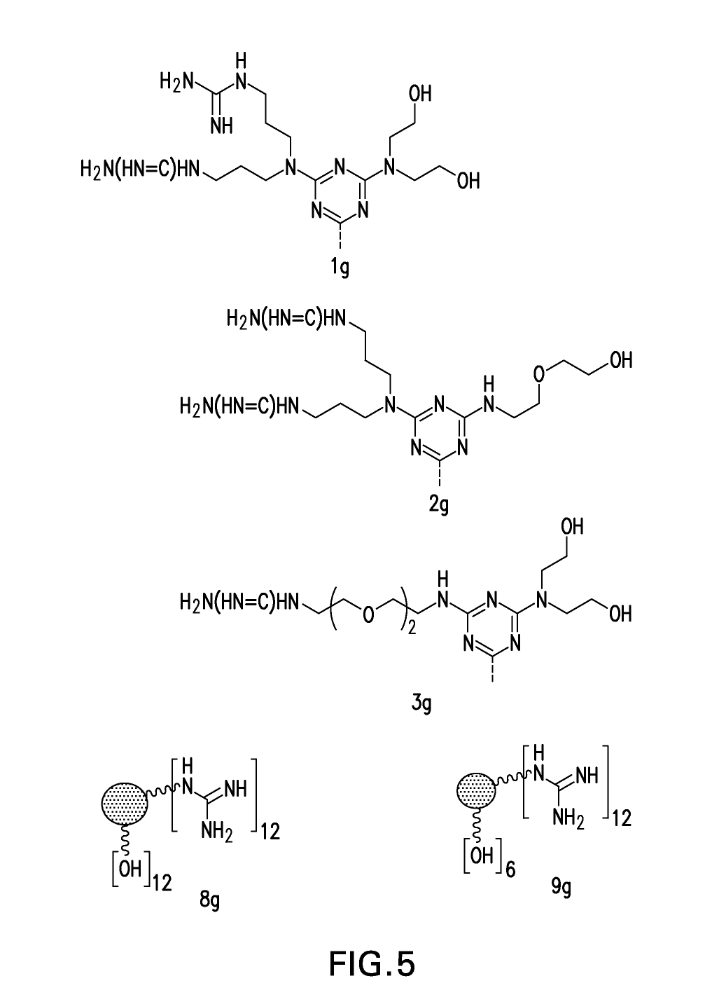

According to still further embodiments, the triazine dendrimer may include guanidium groups. These groups may help promote interactions with cell surface phospholipids and thus improve transfection. In specific embodiments, the guanidium group may be added to a peripheral group. Examples of this for peripheral groups 1-3 are shown in FIG. 5. Throughout this specification, the addition of a guanidium group to a peripheral group is designated by the letter “g” after the peripheral group number. For example, a fourth generation dendrimer X core with peripheral group 1 containing a guanidium group would be designated “X4-1g.”

Additional peripheral groups may also be added to the dendrimer vectors of this disclosure to provide additional functionalities. These moieties may be covalently bonded or they may be attached via other types of interactions, such as electrostatic interactions, and may be directly or indirectly attached to the dendrimer vector. The additional moieties may, if non-covalently bonded or if subject to release in the cell, for example by enzymatic cleavage, may dissociate from the dendrimer after introduction into the cell. These additional moieties may be attached to multiple dendrimer vectors. For example, a large molecule able to avoid glomerular filtration may contain targeting agents as well was multiple dendrimer complexes to facilitate delivery of these complexes to the correct tissue or cells in a patient.

Example additional peripheral groups include carbohydrates, proteins, peptides, ligands, lipids, and small molecules. Additional moieties may include those that promote solubility, reduce toxicity, allow for monitoring distribution, affect targeting, enhance transfection or other efficiencies, or provide an orthogonal route to chemotherapy. In specific examples, these additional moieties may include drugs or imaging agents. They may also include targeting agents to help direct the dendrimer complex to the correct cell or tissue. Other specific examples include: hydroxyls, amino acids, peptides, oligo- or polyethyleneglycol, agents for nuclear imaging, such as MRI or PET, agents for NIR imaging, X-ray imaging agents, cholesterol, aliphatic molecules, aromatic molecules, small molecules such as folate, homing peptides, antibodies or derivatives thereon, FDA approved or experimental drugs, and the like.

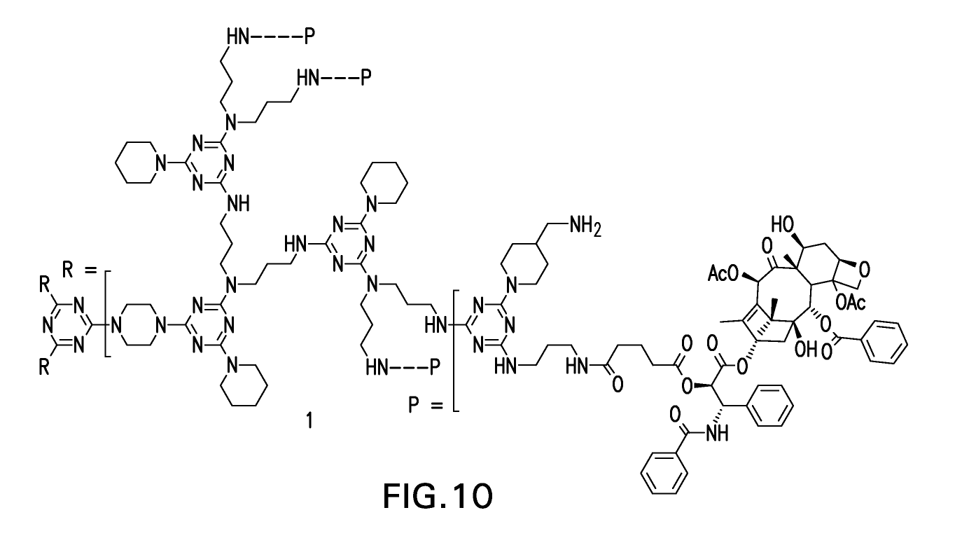

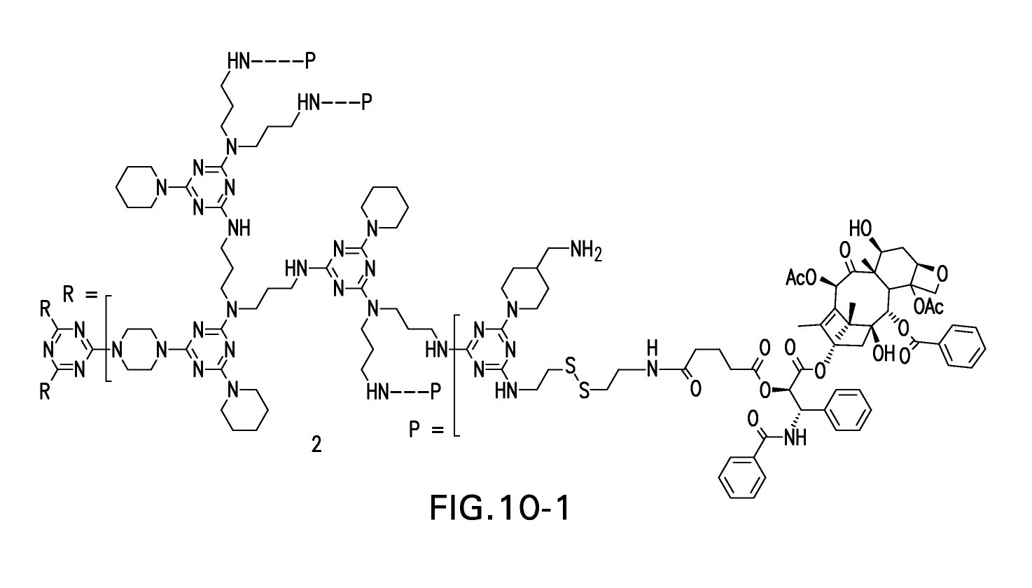

In one specific embodiment, show in FIG. 10, paclitaxel may be attached to a triazine dendrimer as a peripheral group. According to another embodiment, camptothecin may be attached as a peripheral group.

According to specific embodiments, triazine dendrimers of the current disclosure may have molecular weights ranging from thousands to tens of thousands of Daltons.

### Triazine Dendrimer Complexes

The current disclosure also relates to triazine dendrimer complexes containing both the vector and a nucleic acid. The nucleic acid may be bound to the vector in any non-covalent manner that allows it to achieve the desired results of transfection in a cell. In specific embodiments, the nucleic acid may be bound to the triazine dendrimer vector via electrostatic attraction. The type of nucleic acid may include: DNA such as single stranded DNA (ssDNA), double stranded DNA (dsDNA), complementary DNA (cDNA), and the like, as well as RNA, such as single stranded RNA (ssRNA), double stranded RNA (dsRNA), transfer RNA (tRNA), messenger RNA (mRNA), ribosomal RNA (rRNA), small inhibitory RNA (siRNA), antisense RNA, and the like. Nucleic acid mimics may also be used. Nucleic acids may be in any form, such as linear or plasmid. The nucleic acids may contain natural or unnatural derivaties of nucleic acids, including modified nucleic acids, modified nucleosides, synthetic nucleic acids, synthetic nucleosides, or synthetic nucleotides. Nucleic acids may be of any size.

Nucleic acids carried by triazine dendrimer vectors may, in certain embodiment, be intended to result in the expression of a protein and may have both a sequence encoding the protein and regulatory sequences, such as promoters and enhancers. For example, the nucleic acid may include a gene or a gene fragment, including a therapeutic gene or gene fragment. Each complex may contain multiple copies of the nucleic acid.

According to specific embodiments, the triazine dendrimer vector/nucleic acid complexes of the current disclosure may have an average diameter of less than about 200 nm.

Although the embodiments herein focus on the use of triazine dendrimers for transfection with nucleic acids, the triazine dendrimers may be used to non-covalently bind other molecules, such as other polyanions. These bound molecules may be protected or transported into a cell by the triazine dendrimers.

### Selection of Triazine Dendrimers and Complexes

The triazine dendrimer vector selected for particular uses may be determined based on any factors that may influence its suitability for that use. For example, selection may be based on transfection efficiency, hydrodynamic diameter, zeta potential, cytotoxicity, and ease of synthesis. In one example, a melamine-based dendrimer may be used because such dendrimers have low cytotoxicity and are easy to synthesize using divergent strategies that create multiple surface functionalities in a monodisperse manner. Some specific ways to measure these properties and considerations related to these properties that may be taken into account in selecting a triazine dendrimer vector for a particular use are discussed in the below.

In general, triazine dendrimers optimized for a particular function may vary at least in the presence of linkers, the overall size, including generation, flexibility, and solubility.

### Uses of Triazine Dendrimers and Dendrimer Complexes

Triazine dendrimers may be provided as unloaded vectors, for example, as a kit that may also include solutions appropriate for nucleic acid bonding.

Triazine dendrimers may also be provided as complexes loaded with particular nucleic acids. For use in vivo or ex vivo, triazine dendrimers or triazine dendrimer complexes may be provided in a formulation suitable for therapeutic administration or in a kit containing materials to prepare a formulation suitable for therapeutic administration, for example, either as a dried powder or aqueous solution. Formulations may include formulations commonly used for intravenous injection, topical application, intramuscular injection, inhalation, intraperitoneal injection, administration and absorption through a mucosal membrane such as the mouth, or as released components from a matrix (i.e. an eroding hydrogel depot).

In use the triazine dendrimer complexes may contact the target cell and be taken up by the target cell or allow uptake of at least the nucleic acid cargo by the target cell. Potential target cells include bacterial cells, yeast cells, and animal cells, including insect cells, non-human mammalian cells, and human cells. A target cell, in some embodiments, may include an entire tissue, organ, or whole organism.

In some embodiments in which the nucleic acid cargo is intended for gene therapy, the target cell may include, in some embodiments, a foreign cell (e.g. a pathogenic cell in the host organism, or a host cell (e.g. a disease or genetically deficient cell).

### Making Triazine Dendrimers and Complexes

Embodiments of the current disclosure also relate to methods of making triazine dendrimers as described above. In general these methods may derive from a variety of routes. Iterative reaction of dichlorotriazines with a polyamine core, capping, and deprotection may be used. Iterative reaction with a monochlorotriazine with a polyamine core and deprotection may be used. Convergent routes may be used. For example, the triazine peripheral groups 1-5 described above may be added to dendrimers via substitution of BOC-protected monochlorotriazines (the potential location of the chlorine on the triazines in FIGS. 4 and 5 is indicated by the dashed line) through nucleophilic aromatic substitution. The resulting protected dendrimer may be treated with acid to produce the final product.

In another example, guanidine may be added to amine-terminated dendrimer peripheral groups using the commercially available guanidinylation reagent, 1H-pyrazole-1-carboxamidine.HCl.

Nucleic acids may be bound to the completed triazine dendrimer vectors by first purifying the nucleic acid of interest then providing it to the triazine dendrimer vector in a solution that allows bonding. Such a solution may have a particular pH, ionic content, or other properties to allow bonding to form the complex. The solution may differ from physiological or cellular conditions in which bonding is intended to be reversed, allowing release of the nucleic acid after the complex has entered the cell. The solution in other embodiments may be selected so that it may be used during transfection or added to a final formulation containing the complex. In embodiments where the complex will be administered to an organism, the solution may be non-toxic and suitable for the mode of administration, e.g. intravenous injection.

### Examples

The present disclosure may be better understood through reference to the following examples. These examples are included to describe exemplary embodiments only and should not be interpreted to encompass the entire breadth of the invention. Unless otherwise noted, all chemicals used in these examples were analytical grade.

### Example 1

### Effects of Different Dendrimer Properties

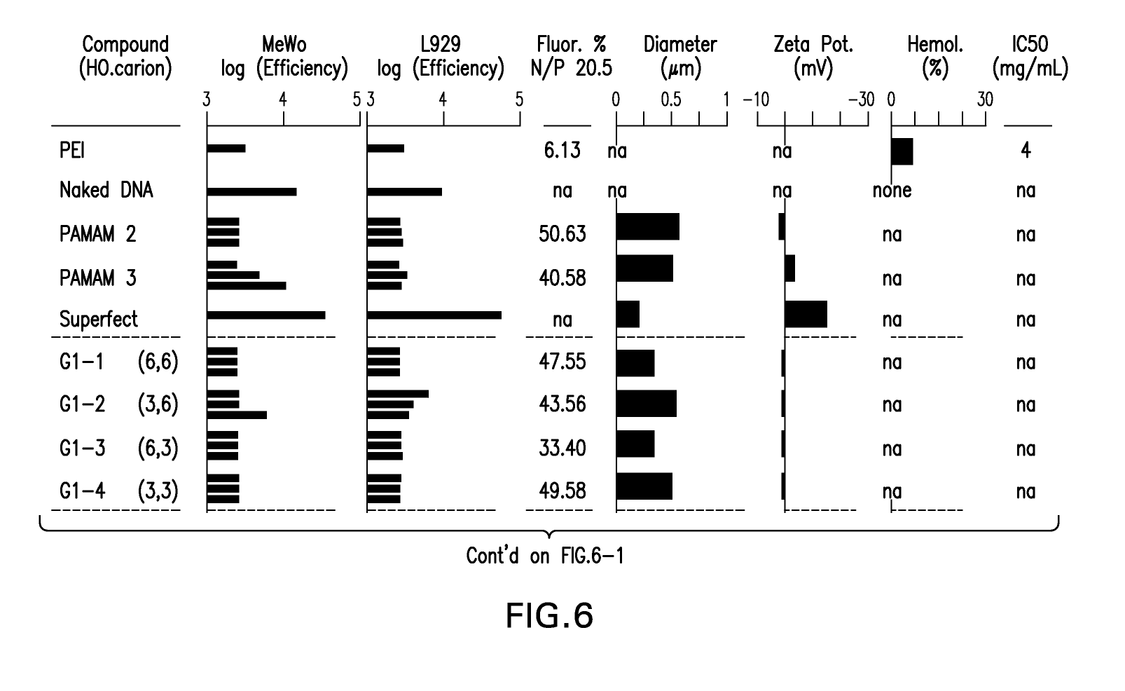

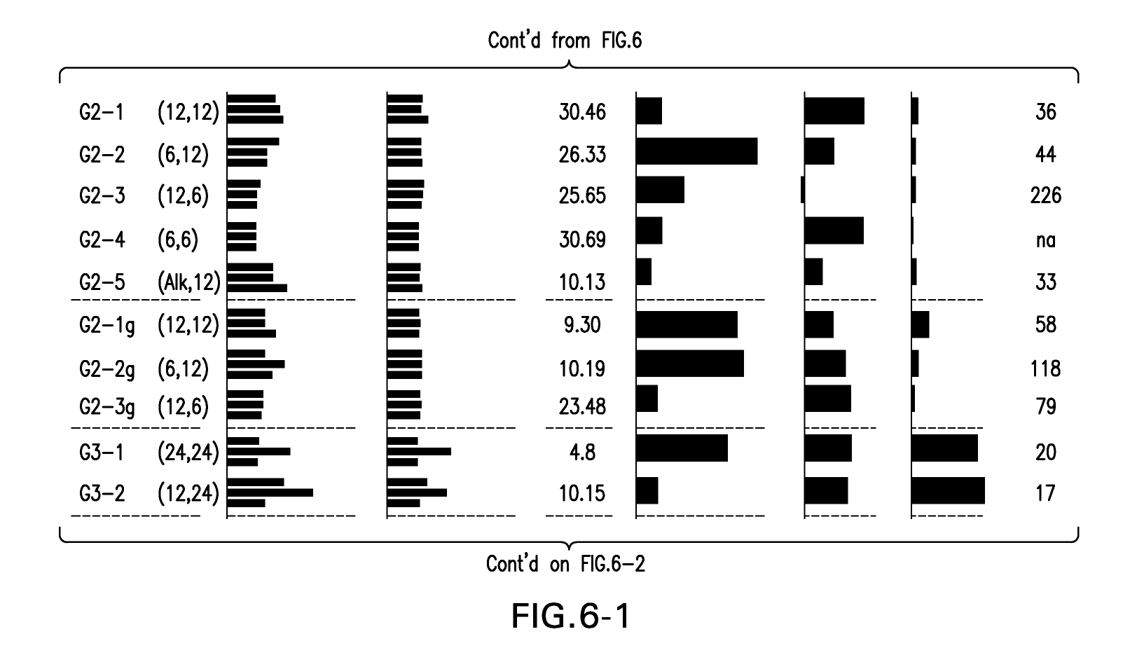

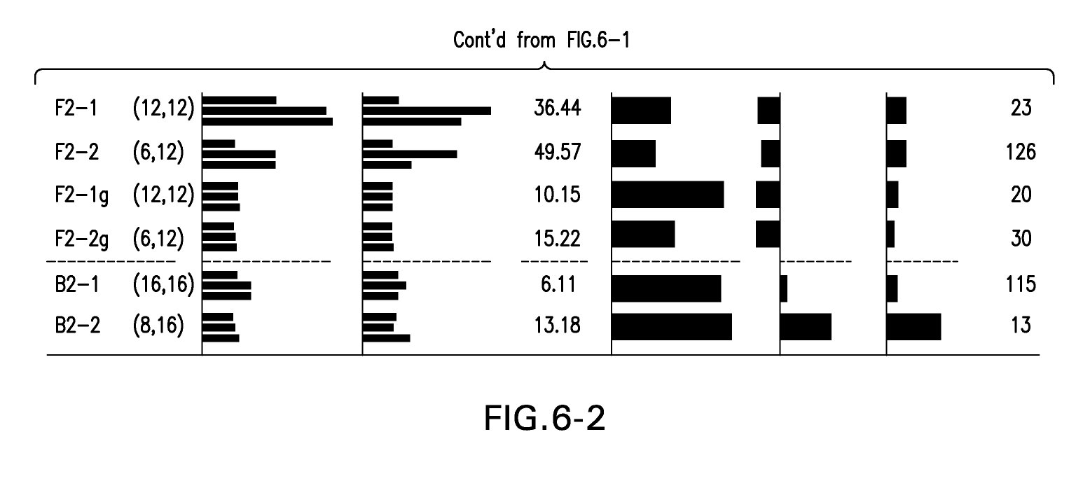

A library of twenty different triazine dendrimers was prepared and assessed using various common assays. For this library, various trends and effects were noted. These effects are described in this example are summarized in FIG. 6. One of ordinary skill in the art may use the effects or similar effects determined using one or more of the same or similar tests to select a particular triazine dendrimer vector for a particular use. Other tests not included in FIG. 6 as well as other properties not tested for in the assays summarized in FIG. 6 may also be used.

The dendrimer core used had a high effect on transfection efficiency, with dendrimer F performing best in this area. Dendrimer F performed as well or better than comparison molecules (PEI, Naked DNA, PAMAM 2 (second generation PAMAM), PAMAM 3 and SuperFect™). Accordingly, flexible triazine dendrimer cores may be selected generally for dendrimer complexes with high transfection efficiency. Fragmented dendrimers, due to increased flexibility, as compared to unfragmented dendrimers, may also exhibit superior transfection efficiency.

Dendrimer generation does not appear to have such a significant effect. Similarly, the identity of the peripheral group also does not appear to have a significant effect for the peripheral groups studied, although some difference could be detected. For all peripheral groups the presence or absence of guanidine did not appear to have a significant effect on transfection efficiency.

The ability of dendrimers to condense DNA related to the dendrimer used, with B performing better than F, which performed better than G. This property also correlated with generation, increasing with higher generations as compared to lower ones, and also with the presence of guanidine, which increased DNA condensation.

### Example 2

### Materials

All reactions were run under atmospheric conditions using reagents as purchased without further purification. Diethanolamine, BOC-anhydride, 2-(2-aminoethyoxy)ethanol, 3,3′-diaminodipropylamine, diisopropylethylamine (DIPEA), and dodecylamine were purchased from Acros Organics (Morris Plains, N.J.). Cyanuric chloride and BOC-piperazine were purchased from Fluka Chemical Corp. (Milwaukee, Wis.). Hexylamine, octadecylamine, 2,2′-(ethylenedioxy)bis-(ethylamine), and 4,7,10-trioxa-1,13-tridecanediamine were purchased from Aldrich Chemical Co. (Milwaukee, Wis.). Dichloromethane, methanol, chloroform, tetrahydrofuran, hexanes, sodium sulfate, and sodium hydroxide were purchased from EMD Chemicals Inc. (Gibbstown, N.J.). Ethyl acetate was purchased from Mallinckrodt Chemicals (Phillipsburg, N.J.). ¹H-NMR spectra were obtained using a Varian Mercury 300 (Palo Alto, Calif.), and chemical shifts (δ) were reported in parts per million (ppm). Mass analyses were carried out by the Laboratory for Biological Mass Spectrometry at Texas A&M University.

### Example 3

### Synthesis of Dendrimers

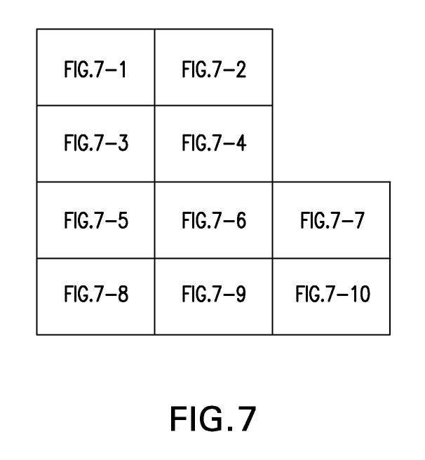

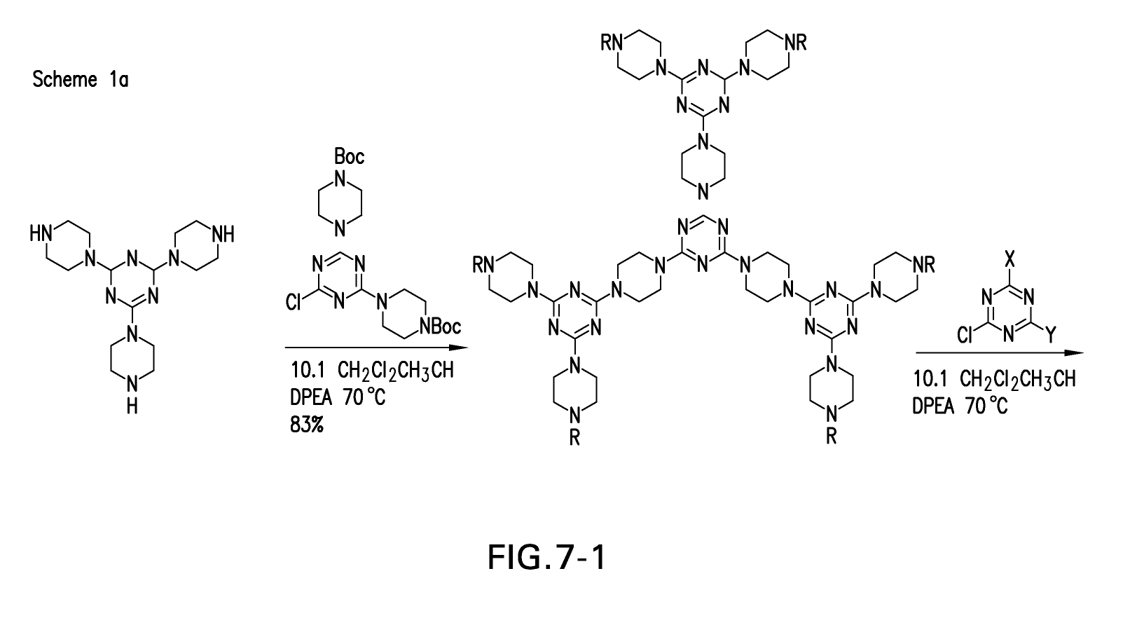

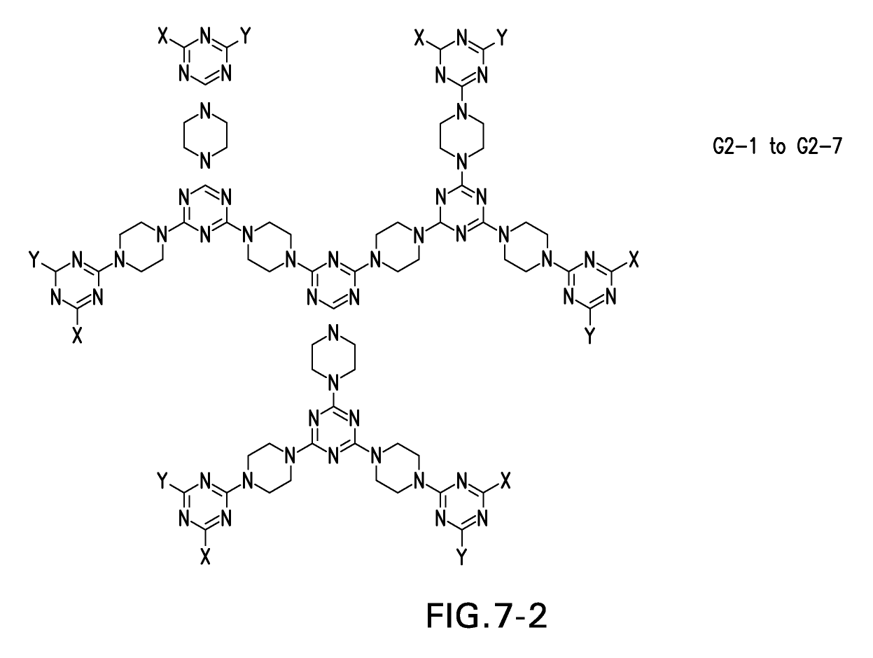

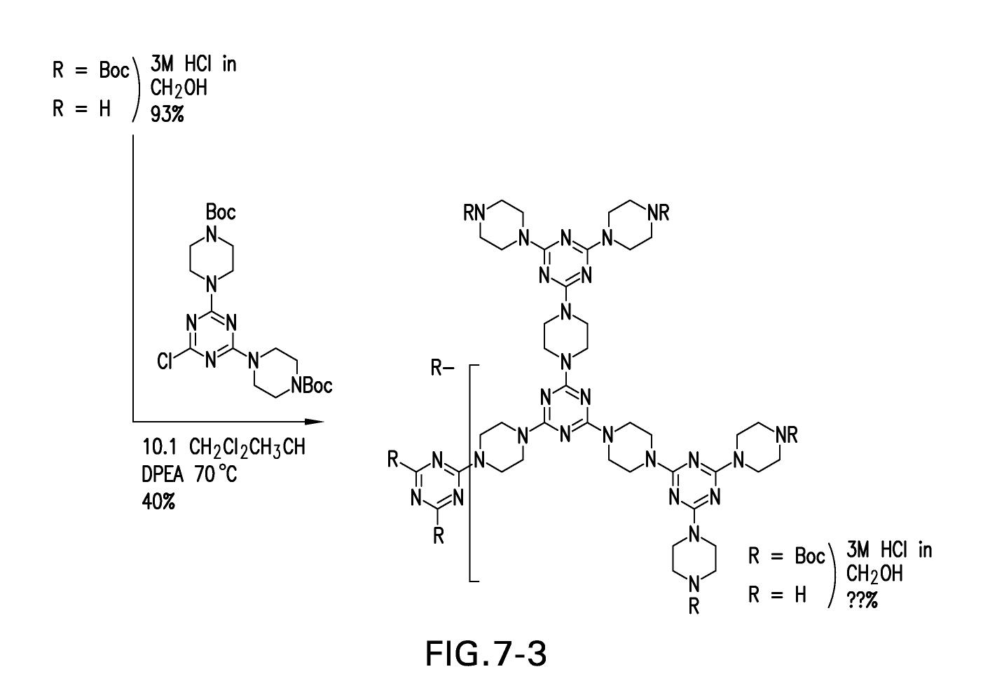

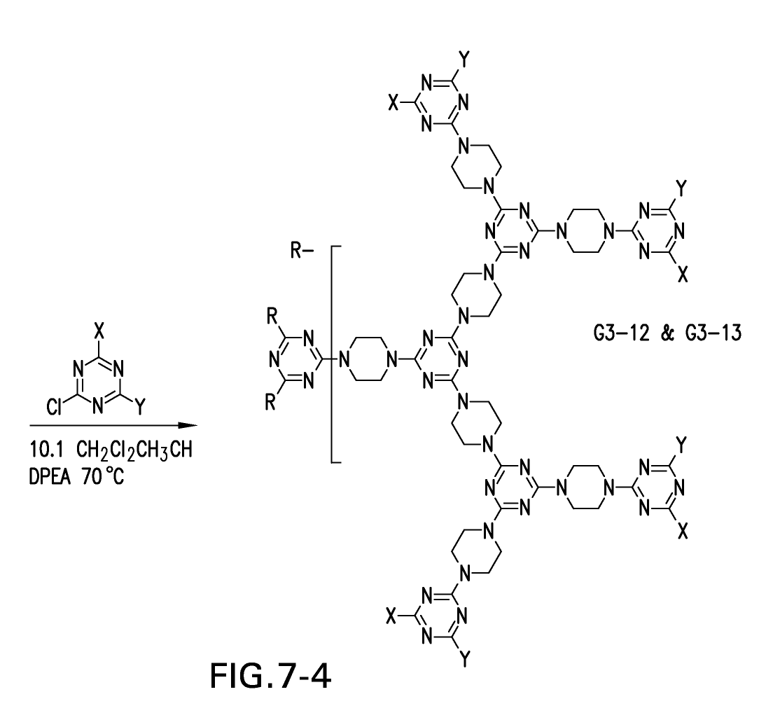

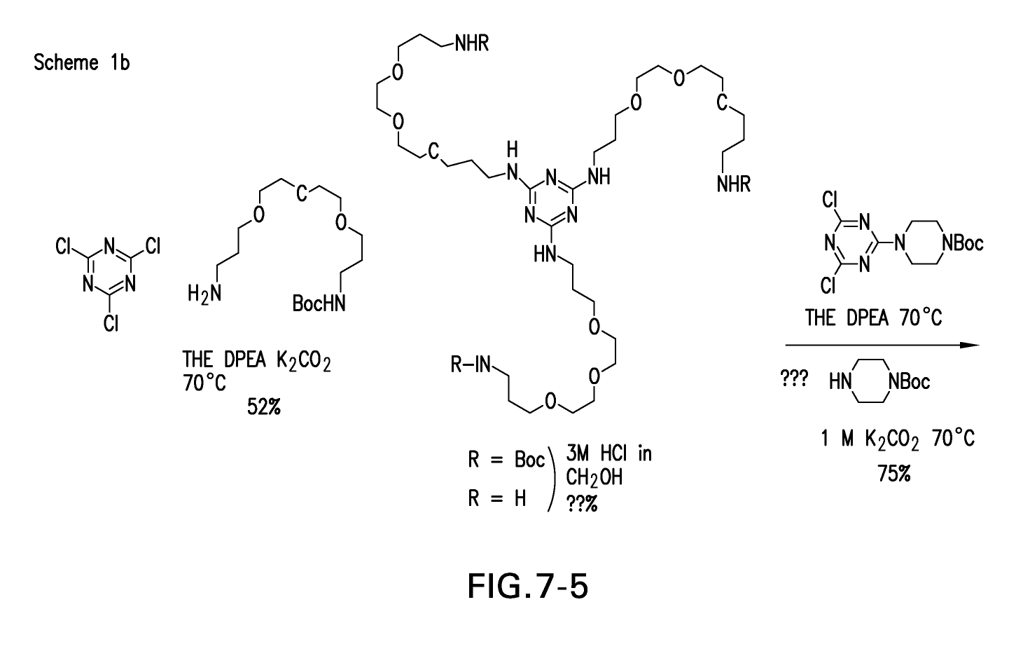

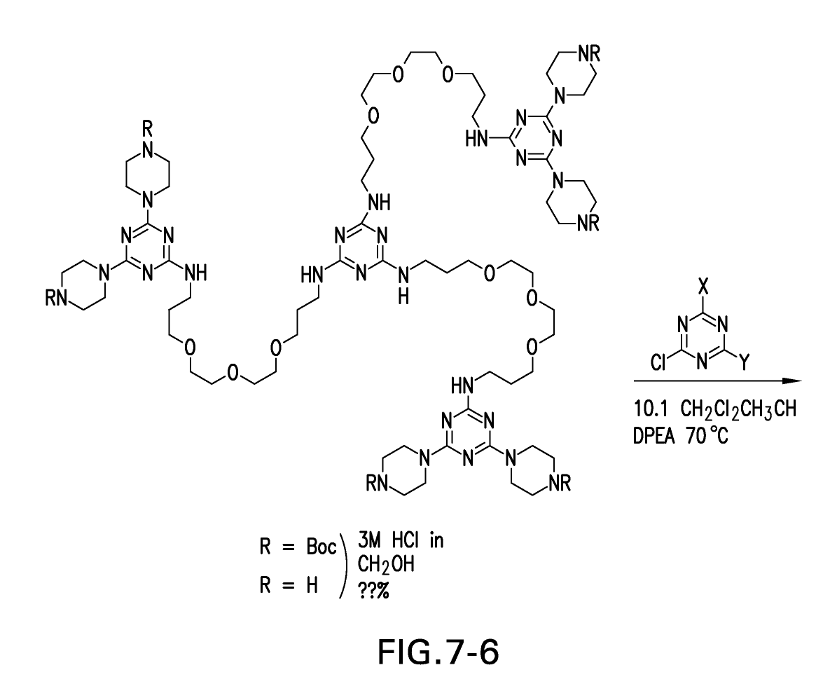

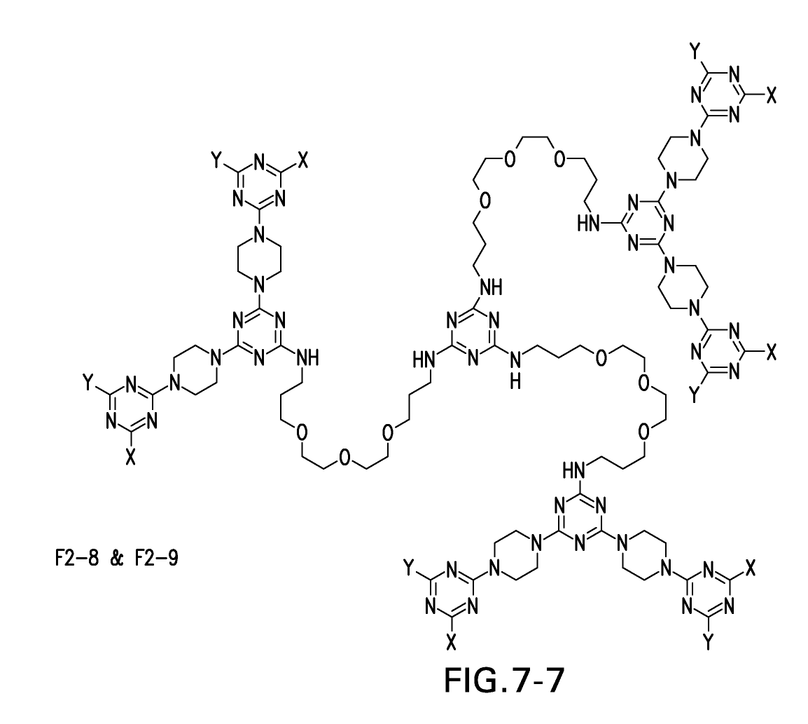

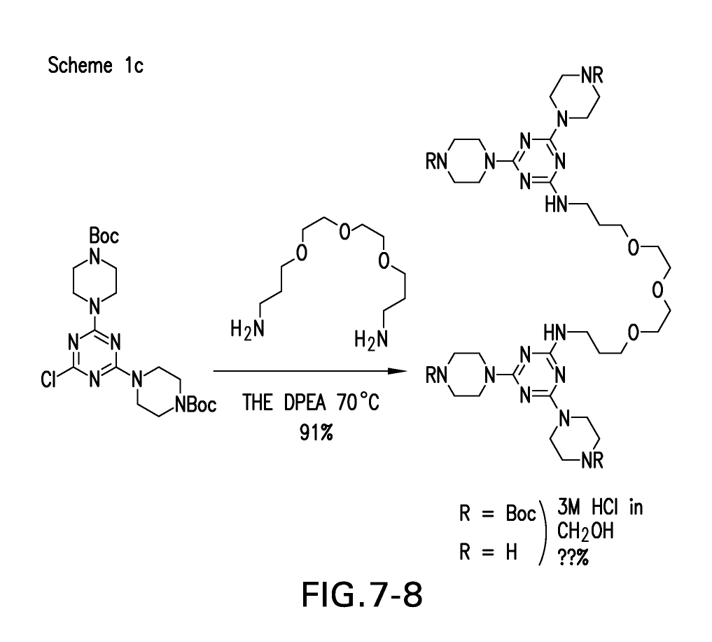

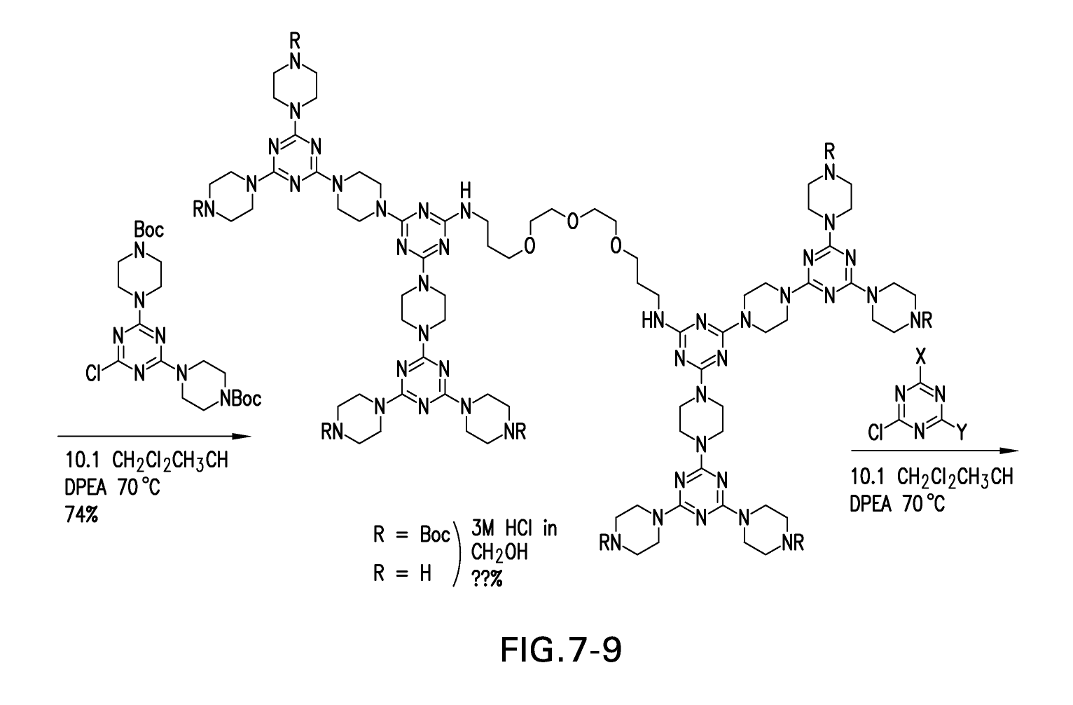

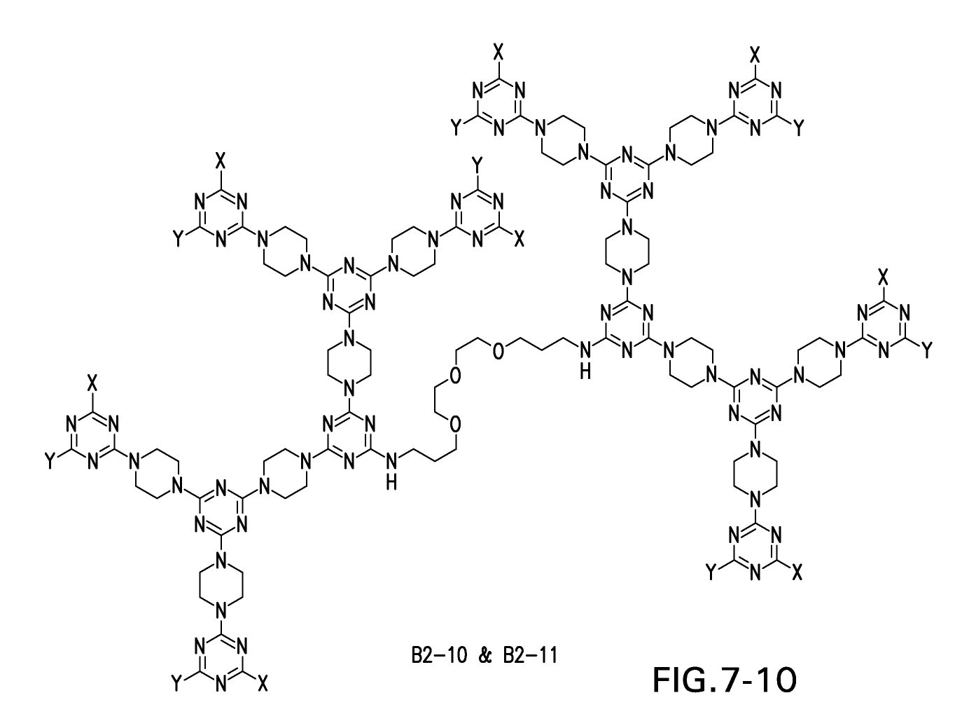

Synthesis of all dendrimers proceeded via a divergent strategy beginning with cyanuric chloride. For the second generation G dendrimers, a tris(piperazyl)triazine core was formed following the protocol described in Chen, H-t, et al., J. Am. Chem. Soc., 126: 10044-10048 (2004). This core was then reacted with di(Boc-piperazyl)monochlorotriazine to form a first generation Boc-protected structure. After deprotection with HCl, the rigid G1 dendrimer was reacted with a variety of protected-monochlorotriazines to form Boc-protected G2 dendrimers, which were deprotected with HCl to and peripheral groups were added to yield compounds G2-1 through G2-7, including guanidinylated variations. This reaction scheme is shown in FIG. 7 (Scheme 1a).

To form the third generation G dendrimers, the rigid, deprotected G1-dendrimer was reacted with di(Boc-piperazyl)monochlorotriazine. This second generation structure was deprotected and reacted with monochlorotriazines to form protected third generation compounds. These compounds were deprotected with HCl to afford compounds G3-1 and G3-2 (Scheme 1a).

The F2 dendrimers were synthesized by reacting cyanuric chloride with mono-Boc-protected O,O′-Bis(3-aminopropyl)diethylene glycol. This core was deprotected with HCl, reacted with Boc-piperazyl-dichlorotriazine, and capped with Boc-piperazine. After removal of the protecting groups with HCl, the compounds were reacted with protected monochlorotriazines to form F2-dendrimers. These compounds were deprotected with HCl and peripheral groups were added to yield compounds generation two F-series dendrimers “F2” with a range of peripheries (F2-1, F2-2, etc.), including guanidinylated variations. This reaction scheme is also shown in FIG. 7 (Scheme 1b).

The B dendrimers were synthesized by reacting di(Boc-piperazyl) monochlorotriazine with O,O′-Bis(3-aminopropyl)diethylene glycol. This core structure was deprotected with HCl and reacted with di(Boc-piperazyl)monochlorotriazine. This first generation bow-tie compound was deprotected with HCl and reacted with protected monochlorotriazines to form the second generation B2 dendrimers. These compounds were deprotected with HCl and peripheral groups were added to yield B-series dendrimers typified by second generation “B2” with a range of peripheries (B2-1, B2-2, etc.) This reaction scheme is also shown in FIG. 7 (Scheme 1c).

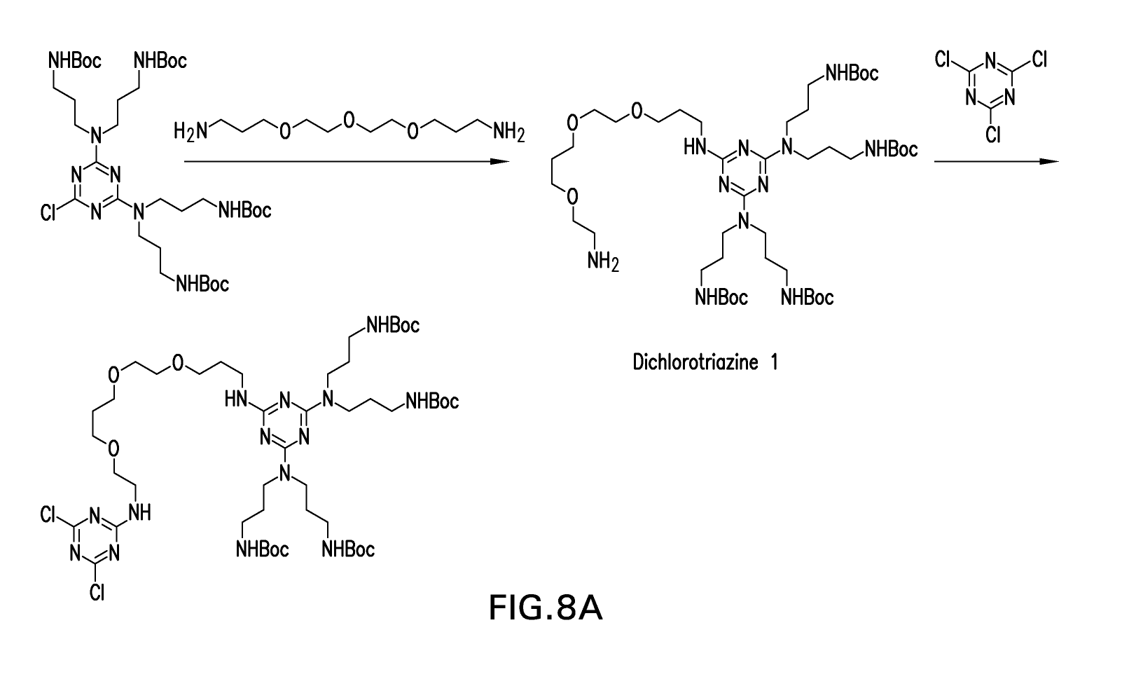

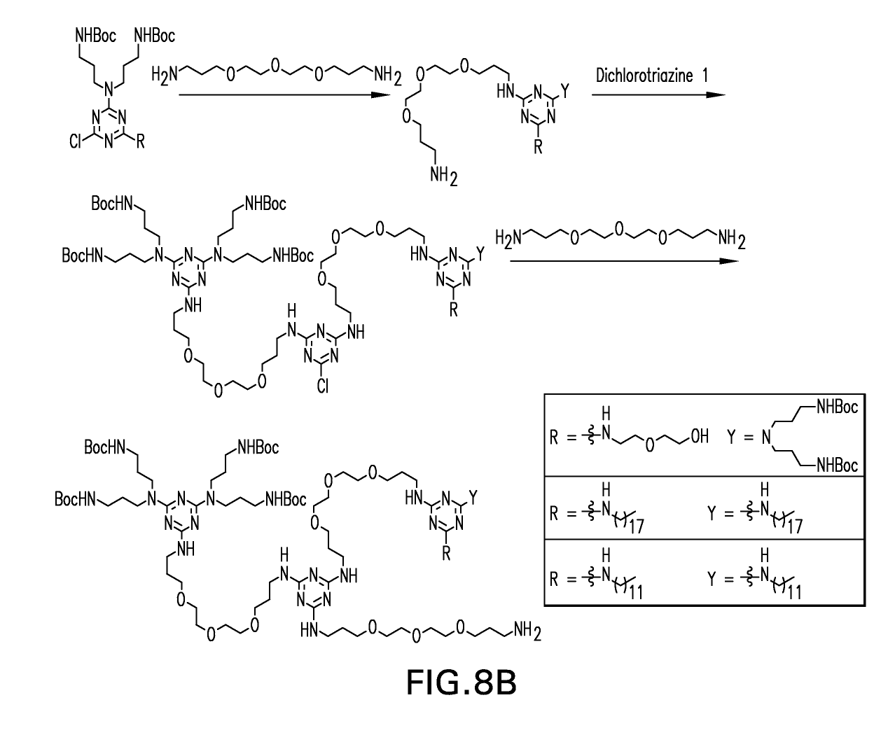

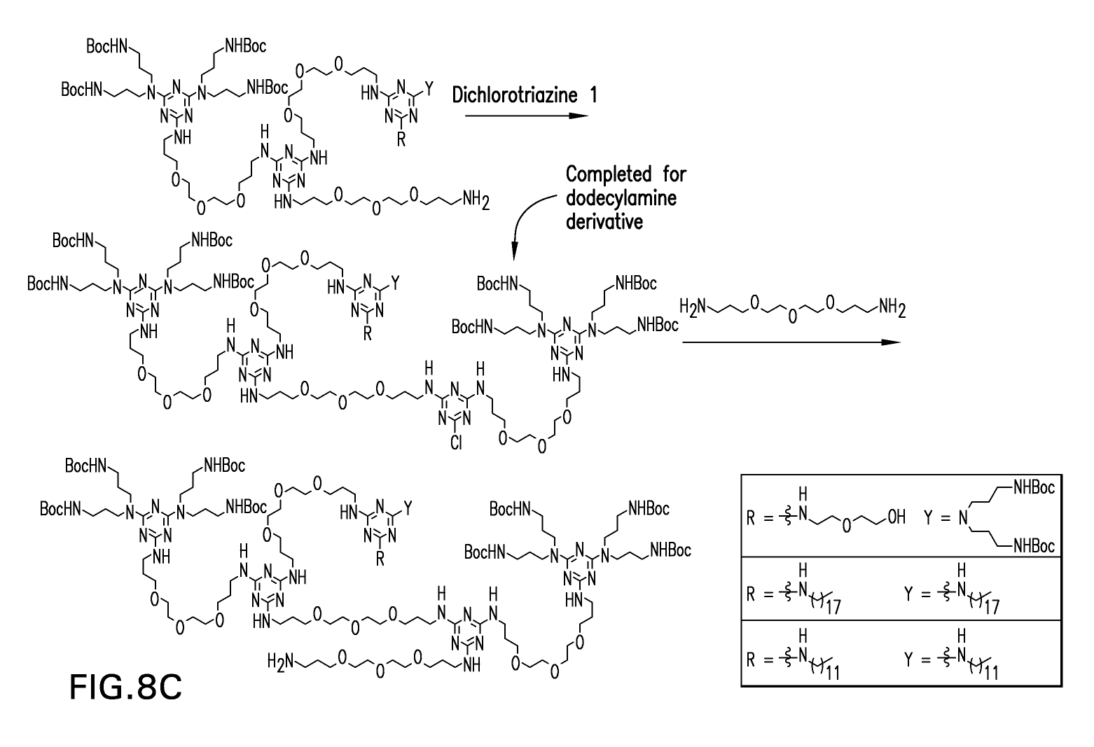

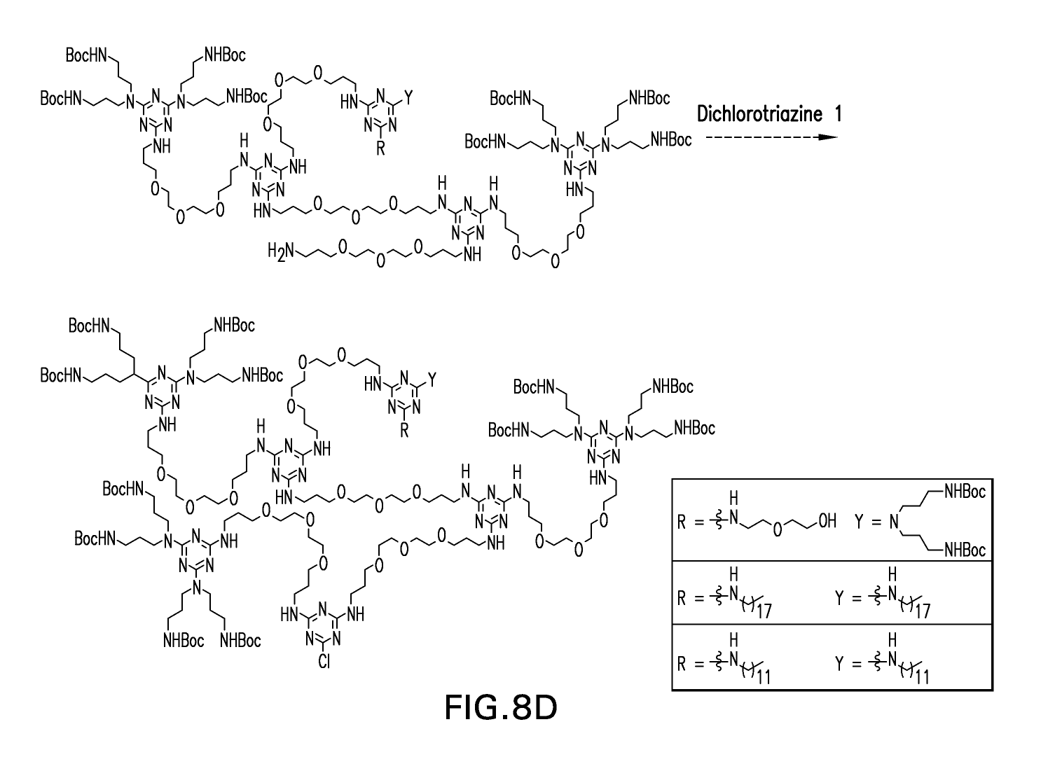

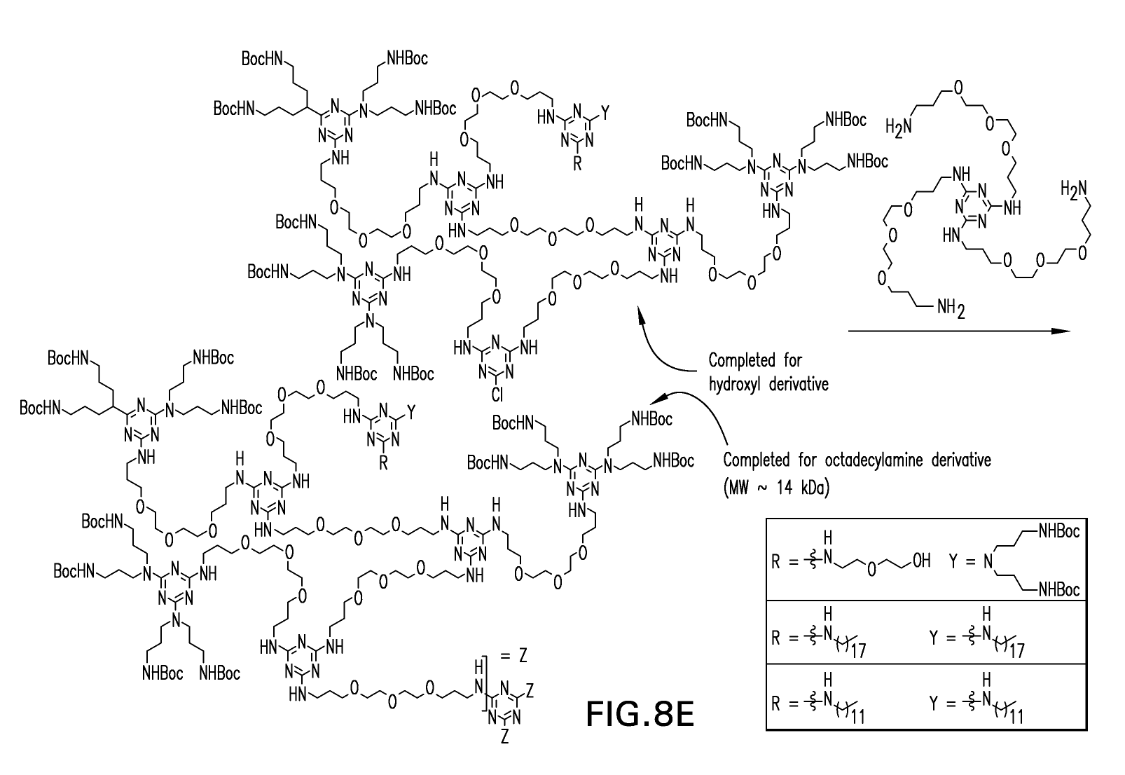

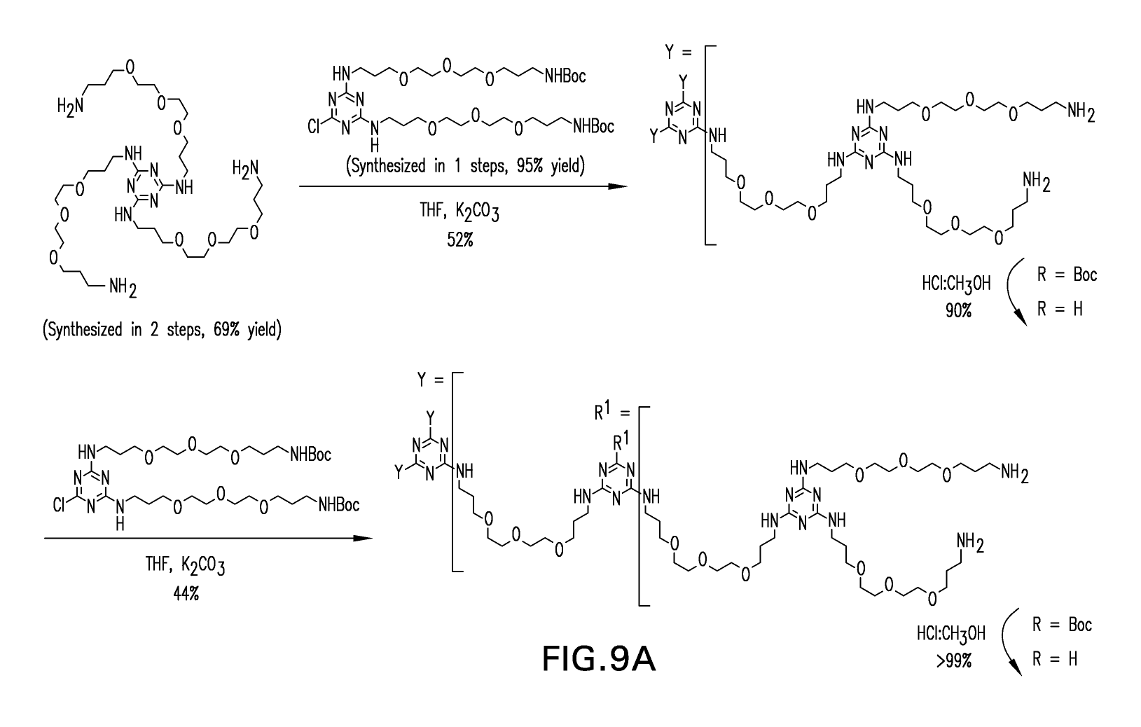

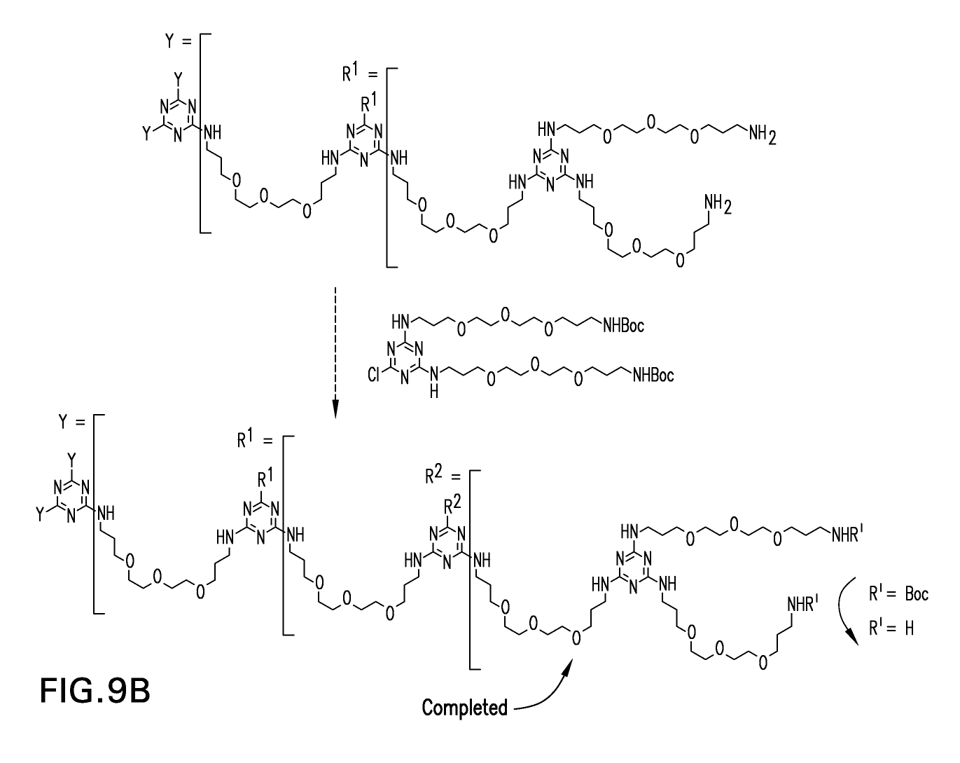

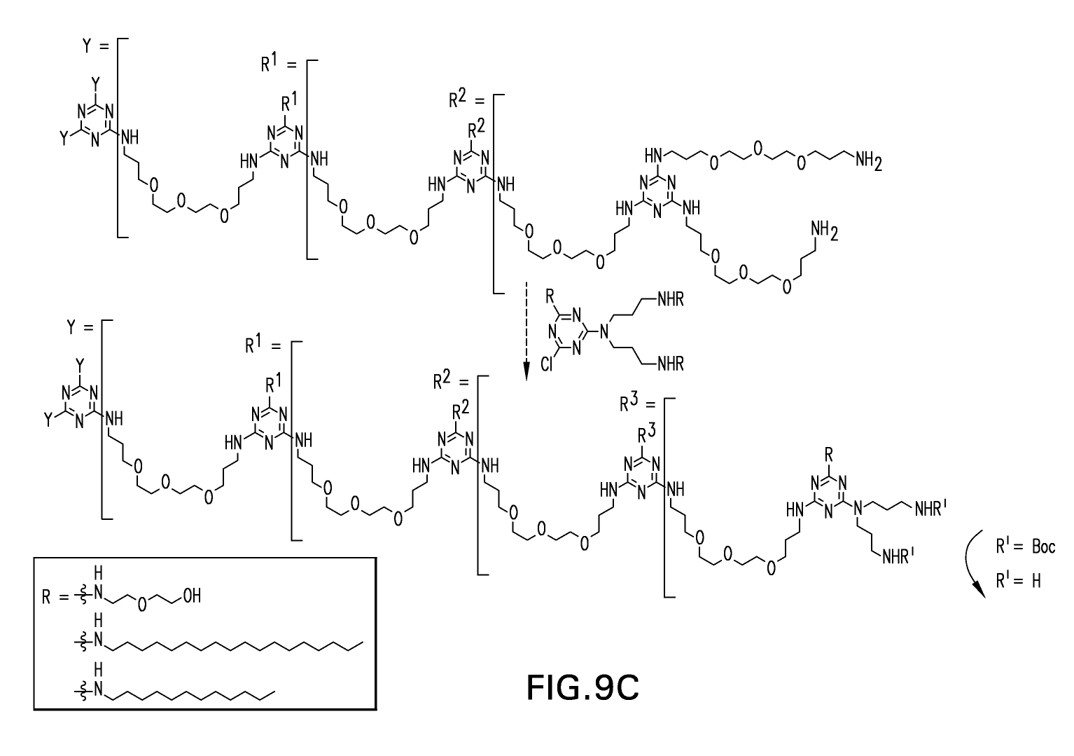

Both fragmented and unfragmented versions of dendrimers may be prepared. One example of alternative synthesis schemes is shown in FIGS. 8 and 9, in which FIG. 8 shows synthesis of a fragmented form of a fourth generation dendrimer, while FIG. 9 shows synthesis of an unfragmented form.

### Example 4

### Preparation of Triazine Dendrimer Vector and Nucleic Acid Complexes

The formation of triazine dendrimer vector and nucleic acid complexes (which may also be referred to as dendriplexes) was accomplished by adding a solution of a specific concentration of triazine dendrimer vector to an equal volume of pDNA solution containing pCMV-Luc (Plasmid Factory, Bielfeld, Germany). The two components were mixed and left for 20 minutes to allow complex formation. Both pDNA and triazine dendrimer vector were diluted with 10 mM HEPES buffer, pH 7.4. The appropriate amount of triazine dendrimer for addition was calculated by considering the desired N/P ratio (the number of nitrogen residues per DNA phosphate) and the protonable unit (mass of dendrimer per protonable nitrogen atom) for each dendrimer.

### Example 5

### Transfection Experiments

Transfection efficiency was measured using 3T3 fibroblasts (L929) and human melanoma cells (MeWo). These cells were seeded in 96-well plates at a seeding density of 8000 cells per well. Twenty four hours later, the cells were transfected with triazine dendrimer complexes containing 0.25 μg pCMV-Luc per well and an amount of vector calculated to be N/P 2.5, 5, or 7.5. Control samples with PEI, PAMAM 2, PAMAM 3, or SuperFect were also transfected. Dendrimer or polymer complexes were added to full serum-containing medium and cells were incubated for 4 hours before the medium was changed. After 44 hours, cells wer washed with PBS, lysed with Cell Culture Lysis Reagent, CCLR (Promega, Mannheim, Germany), and assayed for luciferase expression with a commercial luciferase assay kit (Promega, Mannheim, Germany) on a BMG plate luminometer.

Although only exemplary embodiments of the invention are specifically described above, it will be appreciated that modifications and variations of these examples are possible without departing from the spirit and intended scope of the invention. For example, throughout this specification, lists of alterative materials are often provided. This is not to imply that these materials are the only such materials suitable for the given purpose. Instead, lists are provided to allow one of skill in the art to understand trends and properties of suitable materials and to guide one of skill in the art in selection additional suitable materials if necessary. Similarly, throughout this specification, various modes of action or effects may be described for certain chemical entities. While these modes of action or effects are likely caused by such chemical entities, their description is not intended to imply that other modes of action or effects are not possible or that other different chemical entities may not have different modes of action or effects.

1. A triazine dendrimer complex for transfection of a cell comprising:
a nucleic acid to be transfected, the nucleic acid non-covalently bound to;
a triazine dendrimer vector,
wherein the complex is operable to transfect the cell with the nucleic acid

2. The dendrimer complex according to claim 1, wherein the triazine dendrimer vector comprises a plurality of triazine molecules linked in a symmetric dendritic pattern.

3. The dendrimer complex according to claim 1, wherein the triazine dendrimer vector comprises a plurality of triazine molecules linked in an asymmetric or fragmented pattern.

4. The dendrimer complex according to claim 1, wherein the triazine dendrimer vector comprises a first through eighth generation dendrimer.

5. The dendrimer complex according to claim 1, wherein the triazine dendrimer vector comprises a plurality of triazine molecules linked with a rigid, aliphatic linker.

6. The dendrimer complex according to claim 5, wherein the rigid, aliphatic linker comprises a diazine.

7. The dendrimer complex according to claim 6, wherein the triazine molecules are linked using piperazine according to the formula:

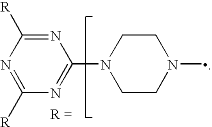

8. The dendrimer complex according to claim 1, wherein the triazine dendrimer vector comprises a plurality of triazine molecules linked with a flexible, hydrophilic linker.

9. The dendrimer complex according to claim 8, wherein the triazine molecules are linked using a flexible, hydrophilic linker according to the formula:

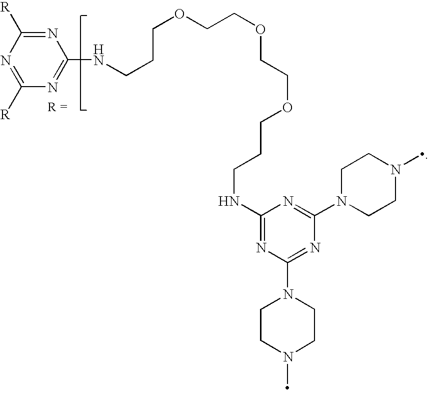

10. The dendrimer complex according to claim 1, wherein the triazine dendrimer vector comprises a plurality of triazine molecules linked with both rigid, aliphatic diamines and flexible, hydrophilic linkers.

11. The dendrimer complex according to claim 10, wherein the triazine molecules are linked according to the formula:

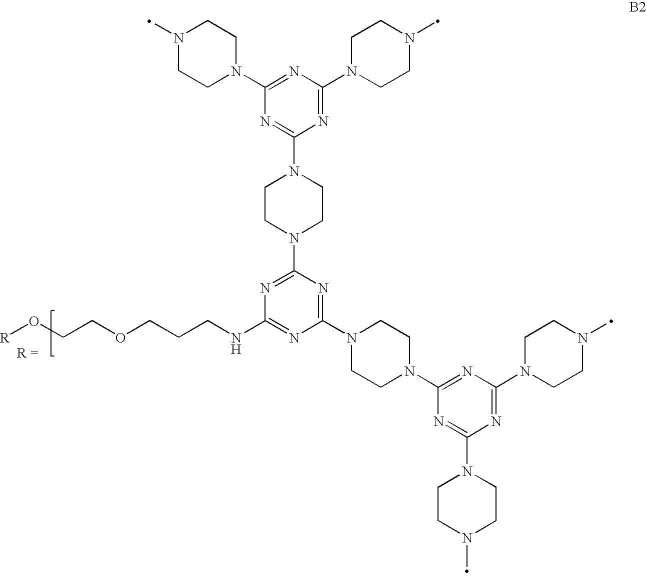

12. The dendrimer complex according to claim 1, wherein the triazine dendrimer vector further comprises a peripheral group.

13. The dendrimer complex according to claim 12, wherein the peripheral group comprises a hydroxyl group, a PEG group, a PEG-like linker group, or an alkyl group.

14. The dendrimer complex according to claim 12, wherein the peripheral group comprises a guanidine group.

15. The dendrimer complex according to claim 12, wherein the peripheral group comprises a drug.

16. The dendrimer complex according to claim 12, wherein the peripheral group comprises a targeting agent.

17. The dendrimer complex according to claim 9, wherein the peripheral group comprises an imaging agent.

18. The dendrimer complex according to claim 1, wherein the nucleic acid encodes a protein or protein fragment.

19. The dendrimer complex according to claim 1, wherein the nucleic acid is operable to interfere with the activity of a nucleic acid in the cell.

20. A method of transfecting a cell comprising:
contacting the cell with a dendrimer complex comprising:
a nucleic acid to be transfected, the nucleic acid non-covalently bound to;
a triazine dendrimer vector,
wherein, after contact, the dendrimer complex enters the cell.

21. A method of preparing a triazine dendrimer complex for cell transfection comprising:
covalently bonding a series of triazine monomers to a central monomer through a series of reactions to form a triazine dendrimer; and
non-covalently bonding a nucleic acid to the dendrimer to form a triazine dendrimer complex,
wherein the complex is operable to transfect a cell.
